(19) United States
(12) Patent Application Publication
(10) Pub. No.: US 2007/0014744 A1
(43) Pub. Date: Jan. 18, 2007

(54) Preparation, in particular cosmetic preparation, process for the production thereof, and use thereof

(76) Inventors: Azra Swistowski, Nuremberg (DE); Christina Zech, Kaufering (DE)

Correspondence Address: BACHMAN & LAPOINTE, P.C., 900 CHAPEL STREET, SUITE 1201, NEW HAVEN, CT, 06510, US

(21) Appl. No.: 11/246,815

(22) Filed: Oct. 7, 2005

(30) Foreign Application Priority Data
Jul. 14, 2005 (DE) 10 2005 033 520.9

(51) Int. Cl. A61K 8/72; A61K 8/73; A61K 8/81

(52) U.S. Cl. 424/70.11; 424/70.13; 424/70.14; 424/70.15

(57) ABSTRACT

A preparation, in particular a cosmetic preparation, in the form of an O/W emulsion, which besides usual cosmetic ingredients, contains a wax component, a fatty acid, a polyvalent alcohol, a film-forming agent and a gel component, wherein the gel component comprises a hydrocolloid.

### BACKGROUND OF THE INVENTION

The invention concerns a preparation, in particular a cosmetic preparation, preferably in liquid or pasty form, for use on the skin, on semi-mucous membranes, on mucous membranes and in particular on keratinic materials such as hair, eyelashes and eyebrows, in particular for shaping, decorating, coloring and improving the appearance thereof and for caring for the skin and the skin appendages. Preparations of that kind are used for example for shaping and coloring in particular the eyelashes and hair; such a preparation is then referred to as ‘mascara’.

Basically preparations according to the invention, with suitable adjustment and coloring, can also be used as makeup, concealer, camouflage, eyeshadow, eyeliner, lipliner, rouge, lip rouge, lip gloss, sun protection agent, sun blocker, temporary tattoo, colored effect sun protection for surfers and the like.

Preparations, in particular cosmetic preparations, with which keratinic material, for example eyelashes or hair can be shaped and colored, have already long been known. Improvements have repeatedly been developed in order to satisfy the many different demands which are to be made on such preparations. Compositions which are applied to hair or eyelashes are on the one hand to provide for good coloring of the hair or eyelashes, but on the other hand they should not transfer on to the surrounding area or other articles such as for example textiles. They should adhere well to hair or eyelashes but not permanently color them and they should also be easy to remove again. It should be possible for them to be applied to the hair and eyelashes easily and uniformly, in particular they should impart an attractive shape to the eyelashes and also retain that shape after drying. For that purpose the compositions should be basically pasty but nonetheless of such low viscosity that they can be applied well and uniformly, but on the other hand they dry quickly, so that the material is not smudged and eyelashes cannot stick together during the drying phase. In addition the preparations should be so stable that they can be stored over a prolonged period of time under ambient conditions. The ingredients thereof should therefore neither separate nor sediment.

To serve the above-mentioned, in part contradictory requirements the most widely varying forms of preparation and ingredients have been proposed. Thus mascara preparations are also known in the form of what is referred to as ‘block mascara’ or ‘cake mascara’—a solid form of preparation from which the material is removed by means of a moistened brush—, in the form of ‘emulsion mascara’, in the form of O/W or W/O emulsions, or in the form of a solvent-based mascara. The use of a ‘block mascara’ is complicated if no water is available for moistening the brush or the material and another common name—namely ‘spit mascara’—shows that alternative forms of use can certainly entail serious microbiologically induced risks in terms of product and user.

Other common mascaras contain a wax component which provides for adhesion to the hair or the eyelash, a film-forming agent which provides for an attractive curved shape for the eyelash and for retaining that shape, and binding agent for adjusting the viscosity in a suitable range. A mascara of that kind then always contains an aqueous phase and a lipid phase. Then—due to the type involved—at least one emulsifier must always be used in such an ‘emulsion mascara’ in order to keep the continuous phase in intimate relationship with the dispersed phase. A disadvantage with such a product is generally that, after drying on the eyelashes, it can be dissolved again due to moisture from the environment, for example water, rain, perspiration and in particular the quite slightly alkaline tear fluid, whereby the durability of such preparations is reduced. The use of water-soluble polymers or polymers which can be dispersed in water also involves detrimental effects here. An example of such a polymer-bearing product is to be found in W Umbach, ‘Kosmetik’, Georg Thieme Verlag, Stuttgart, 1988, page 101. Other examples are to be found in the standard works in cosmetics literature, for example H Janistyn, ‘Handbuch der Kosmetika und Riechstoffe’, volume 3, Hüthig-Verlag, 2nd edition, 1973, on pages 855-860 or G A Nowak, ‘Die kosmetischen Präparate’, Verlag Ziolkowsky, 3rd edition, 1984, pages 748-751.

In addition for example U.S. Pat. No. 6,210,692 describes cosmetic emulsions which are suitable inter alia as mascara and which are made up of an aqueous phase and a lipid phase, which, for stabilisation purposes, contain two different binding agents, namely a hydrophilic thickening agent and a polysaccharide ether, in which respect the use of such thickening agents which form gels with the aqueous phase is to be avoided. Those emulsions are said to be particularly suitable for caring for and treating dry sensitive skin. In addition U.S. Pat. No. 6,325,994 describes a cosmetic preparation which is suitable in particular as mascara and which comprises a lipophilic phase with fat and wax, as well as a lipophilic polymer, that lipophilic polymer being a specific acrylate-methacrylate copolymer. That composition can also occur in the form of an emulsion with an aqueous phase. Furthermore that composition may contain conventional additives such as coloring agents, pigments and fillers. Although that composition is said to be water-resistant and very durable it is still not satisfactory in every respect.

The comparable compositions described in the above-mentioned literature usually contain lipids such as for example waxes and oils, emulsifiers, binding agents and coloring agents and they occur in emulsion form—either as an oil-in-water emulsion or a water-in-oil emulsion. The known preparations however suffer from at least one of the disadvantages referred to hereinafter. Many of the known emulsions are not sufficiently stable to be processed in the heated condition. For cosmetic compositions however a heat treatment may be required, which then necessitates specific and complicated and expensive treatment stages. Because of their nature, it is frequently different to incorporate coloring agents, in particular pigments, in such a way that they remain stable in the composition without settling out or forming lumps. Compositions which have good adhesion to keratinic materials are often difficult to remove therefrom again. In particular water-resistant and tear-resistant compositions can no longer be removed by simple washing with water and they then require the use of special ‘makeup removers’.

In addition, a particular disadvantage with compositions of that kind is that, after drying on eyelashes or hair, they often form a brittle coating which can tear and flake off. In the case of hair mascara, that effect can certainly be desired because such a preparation, in the form of temporary hair coloring, can later be easily removed again by brushing it out—if however fine particles flake off the eyelashes, they can land in the eye and result in mechanical irritation, which for example in the case of ladies wearing contact lenses, can also discolor the lenses; however the region under the eye can also be colored (known as what is referred to as the ‘panda bear effect’).

Solvent-bearing mascara preparations are also known, which contain only little or no water at all. Polar solvents such as ethanol, propan-1-ol, propan-2-ol, acetone, methylethylketone, ethylacetate, tetrahydrofuran, dichloromethane and the like are admittedly recommended in earlier technical literature—in that respect they automatically rule themselves out by virtue of their odor, their severely irritating effect on skin and mucous membranes and lack of environmental compatibility. Ethanol can at best be used in amounts below about 15% by volume. Non-polar isoparaffins have admittedly already been used in the past—in regard to some thereof, a point which tells against them is the aromatics content, which excludes them for cosmetic uses and the easy flammability of the low-boiling isoparaffins, whereby specific precautions are advised in the production of preparations of that kind. If however exclusively or predominantly higher-boiling C₁₁/C₁₂ isoparaffins are chosen, preparations which adhere very well to skin and hair are admittedly obtained, which have very good transfer resistance—however they then generally have very long drying times, which makes them markedly uncomfortable from the point of view of users. A further disadvantage is also that diffusion problems and stress crack corrosion can occur when using containers of given kinds of polypropylene.

### Statement of Object

Therefore the inventors of the present application set themselves the objects of developing a product which is simple to manufacture and which can also be safely and reproducibly manufactured without special machine plant; into which pigments can be easily incorporated and remain stable therein; which affords a good uniform coloring effect and which nonetheless is not overburdened with pigments; which is free of non-ionogenic emulsifiers based on polyoxyethylene (PEG) or polyoxypropylene (PPG) as they are judged to be a source of irritation in the proximity of the eye; which is stable even at elevated temperature and which can therefore be heated without separation or destruction; which at elevated temperature is of such a viscosity that it can be easily filled into containers; which can be applied easily and uniformly, which adheres for a prolonged period of time, which can extend the eyelashes, which can impart bulk and volume to the eyelashes, which is forgiving in terms of application mistakes and allows subsequent corrections and which even after hours allows makeup touching-up in order to make the appearance more intensive, which does not transfer on to eyelid surfaces, which is water-resistant and tear-resistant, which does not flake off or crumble away after drying and which can nonetheless be easily removed again with normal means.

### SUMMARY OF THE INVENTION

In accordance with the invention all those objects are attained by a preparation, in particular a cosmetic preparation, which is in the form of an oil-in-water emulsion (O/W emulsion) and which besides usual cosmetic ingredients contains at least one wax component, a fatty acid, a film-forming agent, a polyvalent alcohol, a hydrocolloid as a gel component and a non-ionogenic emulsifier which has no PEG or PPG residues.

### DETAILED DESCRIPTION

It was surprisingly now found that the use of sorbitan olivate in combination with at least partially neutralised stearic acid results in very stable O/W emulsions enjoying particularly good temperature stability, in which coloring agents, in particular pigments, can be very easily and uniformly distributed, which can then no longer migrate out of that material, so that the applied material no longer runs even upon contact with water, perspiration or tear fluid. The structure of that O/W emulsion is so stabilised in the solid and also pasty condition that no separation of the ingredients thereof occurs even at higher temperature. That affords the advantage that it can be heated without any problem and in the heated condition and thus at lower viscosity can be filled into containers, which facilitates and speeds up the production and filling process. Production of the preparation according to the invention is also simple and can be easily and securely reproducibly effected with conventional available items of equipment. In that respect the use of a vacuum process installation is particularly advantageous, allowing the uniform removal of air from the preparation for further improving storage stability.

The preparation and in particular the cosmetic preparation is in particular intended to be applied to keratinic material, in which respect consideration is given in particular to eyelashes, eyebrows, hair and hair pieces. It is also possible for the preparation according to the invention to be used for men for hiding hair which has turned gray at the temples or for dying beards. The preparation according to the invention is particularly suitable in the form of a mascara.

The most important constituents of the preparation according to the invention, besides the water forming the continuous phase of the emulsion, are a wax component, a polyvalent alcohol, a film-forming system and a gel component.

The wax component comprises at least one wax and possibly additionally at least one fat and/or oil which can respectively be of vegetable, animal, mineral or synthetic origin. In addition the composition can contain at least one emulsifier and at least one co-emulsifier to facilitate processing of the wax component to afford an emulsion. In order to achieve a result which is particularly satisfactory from the esthetic point of view, the wax component can additionally contain a polyvinylpyrrolidone copolymer.

The wax component imparts the desired consistency to the material and makes the composition water-resistant and tear-resistant. For that purpose the wax component can be made up from fat-like, oil-like and wax-like raw materials which can be fluid, pasty or solid at room temperature—that is to say at 23° C.±2° C. Preferably a combination comprising at least one wax and at least one oil is used for adjusting the optimum consistency.

Preferably at least one wax which is solid at room temperature is used. The waxes are selected from those of vegetable, animal, mineral or synthetic origin or hybrids. Waxes with a dropping point of between 45 and 200° C. and of a hardness of between 2 and 40 are particularly preferred. Waxes with a dropping point below 45° C. can cause problems in terms of storage capability while waxes with a dropping point above 200° C. are in part difficult in terms of processing. The hardness of the waxes is determined using the needle penetration method. The operation of determining the wax hardness is effected in accordance with the American Standard ASTM D5: at a temperature of 25° C. a needle with a defined cone and weighing 2.5 g which is loaded with a weight of 47.5 g is caused to penetrate into a flat surface of a test body. The hardness value is determined as the depth of penetration in tenths of a millimeter after 5 seconds.

The waxes usually employed in cosmetics are suitable for the wax component used according to the invention, in particular vegetable waxes such as carnauba and candelilla wax, ouricuri wax, Japan wax, cotton wax, rice wax, flower waxes and/or fruit waxes such as orange flower wax, orange wax, jasmine wax or apple wax, hydrated jojoba oil; animal waxes such as beeswax, modified beeswax, inter alia so-called ‘cera bellina’ (polyglyceryl-3 beeswax), lanolin waxes and insect waxes; mineral waxes such as montan waxes, ozocerite, paraffin waxes and microcrystalline waxes, as well as synthetic waxes such as Fischer-Tropsch waxes, wax-polymer hybrids, polyethylene waxes, silicone waxes, long-chain esters such as cetyl palmitate, cetyl stearate, behenyl stearate, behenyl oleate, behenyl behenate, acelaic acid dioleylester, acelaic acid dibehenylester and mixtures of all the listed waxes. Vegetable waxes and synthetic waxes as well as in particular mixtures thereof are particularly preferred.

The wax component includes at least one oil or fat in order to adjust the viscosity of the preparation to the desired range and to impart malleability to the preparation. Considered for that purposes are oils and fats which can be of vegetable, animal, mineral or synthetic origin and which can also be used in hydrated or modified condition. Examples of suitable oils and fats are rapeseed oil, sunflower oil, sesame oil, ground nut oil, thistle oil, coconut oil, hydrogenated coconut oil, castor oil, hydrogenated castor oil, neat's foot oil, beef tallow, isopropyl myristate, isopropyl palmitate, isopropyl stearate, butyl stearate, isobutyl stearate, isostearyl-isostearate, oleyloeate, oleylerucate, diethyl sebacate, hexyl laurate, dibutyl adipate, caprylic/capric triglycerides and similar synthetic triglycerides, paraffin oils, polybutene, squalane, squalene, and mixtures thereof, synthetic esters, silicone oils, in which respect here both volatile and also non-volatile silicone oils are considered, dimethicone (dimethylpolysiloxane), alkyl dimethicone whose alkyl residues can have chain lengths of between 10 and 36 carbon atoms, bis-PEG/PPG-16/16 PEG/PPG-16/16 dimethicone, diphenyl dimethicone, phenyl trimethicone, cyclomethicone, as well as volatile and non-volatile paraffins and/or isoparaffins. Mention will be made here by way of example of straight chain volatile silicone oils such as hexamethyl disiloxane, octamethyl trisiloxane, decamethyl tetrasiloxane and/or decamethyl pentasiloxane and/or cyclic silicone oils such as hexamethyl cyclotriloxane, octamethyl cyclotetrasiloxane, decamethyl cyclopentasiloxane and/or decamethyl cyclohexasiloxane. Among the volatile paraffins and isoparaffins, mention is to be made of those with between 10 and 16 carbon atoms, in particular decane, isodecane, dodecane, isododecane, tetradecane and/or isotetradecane. In that respect C₁₁/C₁₂ isoparaffin is particularly advantageous. In that respect the use of mixtures of the specified substances is particularly advantageous. Thus a non-volatile oil or fat and also a volatile oil is frequently contained in the composition in order to impart various properties to the preparation. In one embodiment the wax component contains volatile constituents, in particular dimethicone and/or cyclomethicone and/or isoparaffins in a concentration of up to 20% by weight with respect to the total weight of the preparation. In an individual case the amounts involved can certainly slightly rise above or fall below the quantitative specifications set forth here and also hereinafter and in that case preparations according to the invention can nonetheless be obtained. For the man skilled in the relevant art, that would in no way be unexpected, in consideration of the large number of specified possible substances—he would know therefore that the fact of the amounts involved being above or below the specified quantities would not entail a departure from the scope of the above-specified invention.

As mentioned, preferably mixtures of waxes, oils and fats are used, wherein the respectively listed substances are used in such amounts that the desired properties such as structure and viscosity are achieved. The respective mixtures and amounts to be used are known to the man skilled in the relevant art and therefore do not need to be discussed in greater detail.

The wax component can in addition have still further constituents which are basically known to the man skilled in the art and which influence properties such as stability, viscosity, processibility and durability. In that respect, raw materials such as medium-chain to long-chain saturated, branched-chain or straight-chain fatty alcohols or fatty acids such as myristyl alcohol, cetyl alcohol, stearyl alcohol, behenyl alcohol, ceryl alcohol, myricyl alcohol, myristic acid, palmitic acid, stearic acid, arachic acid, behenic acid, cerotic acid, melissic acid or mixtures of the specified substances are comparable to waxes in respect of their properties.

Preferably the above-described wax component is used in such a proportion that it forms between 0.5 and 50% by weight, preferably between 12 and 30% by weight, of the finished composition, the percentages always relating to the amount by weight in the finished composition. Within the wax component the proportion of the wax is preferably between 20 and 100% by weight and is particularly preferably between 60 and 90% by weight.

The oil or fat is usually added in such a proportion that the consistency of the material is in the desired range. Normally the proportion of the oils or fats in the wax component, depending on the other materials used, is between 20 and 80% by weight, in which respect the lower range is preferred, and with the proviso that the total of the percentages, with respect solely to the ingredients of the wax components, does not exceed 100% by weight.

In addition, as already mentioned above, the wax component may also contain emulsifiers and co-emulsifiers. They promote emulsion formation and homogeneity of the preparation and stabilise the emulsion formed. Non-ionogenic emulsifiers can preferably be used here as anion-active, cationic or amphoteric emulsifiers result in irritation in the region of the eye because of their salt-like character, in particular if they come into prolonged contact with the mucous membranes of the eye. In particular the alkali salts of fatty acids—also as co-emulsifiers—have proven to be less suitable in that respect as the complete saponification thereof occurs at pH-values of between 9 and 10, which can result in irritation when acting for a prolonged period of time on the mucous membranes of the eye. Salts of organic bases with fatty acids are already formed in the weakly alkaline range, but certain organic bases can result in markedly perceptible and unpleasant secondary odors in a preparation. In accordance with more recent discoveries however non-ionogenic emulsifiers which have PEG or PPG components can also result in irritation in the region of the eye. Therefore O/W emulsifiers based on sorbitan have proven to be particularly suitable for the preparations according to the invention, in particular so-called sorbitan olivate, the monoester from the fatty acids of olive oil with sorbitol, which naturally does not have any PEG or PPG components. In that respect the combination of sorbitan olivate with saturated, straight-chain or branched-chain fatty acids, at least partially neutralised with organic bases, of chain lengths of between 14 and 24 carbon atoms, was found to be particularly advantageous, in which respect here stearic acid, isostearic acid or behenic acid, at least partially neutralised with aminomethyl propanol (AMP) has been found to be particularly advantageous. Stearin neutralised with triethanolamine, the commercially available mixture of palmitic and stearic acid, was also found to be suitable as a co-emulsifier, if certain odor problems are accepted in that respect, with a cheapening effect. In that respect, a quantitative ratio of sorbitan olivate to stearic acid, which is then at least partially neutralised with aminomethyl propanol, of between 3 and 1 to between 1 and 3 was found to be particularly suitable.

It was surprisingly found that the use of a PVP copolymer such as PVP/hexadecene copolymer or PVP/eicosene copolymer or a mixture of the two copolymers in conjunction with sorbitan olivate also has an emulsion-stabilising effect. In that situation of use the copolymers act not only as film-forming agents and dispersing additives for solids but surprisingly also as a co-emulsifier. It is advantageous in that respect to select a quantitative ratio between sorbitan olivate and PVP copolymer in the range of between 10 and 1, to between 3 and 1.

A further essential constituent according to the invention is a polyvalent, at least divalent alcohol which optionally can also have further functional groups, for example amino functions. Examples of suitable divalent or polyvalent alcohols are propane diol, dipropylene glycol, tripropylene glycol, glycerin, diglycerin, triglycerin, butane diols, pentaerythritol, hexane diols, sorbitol, xylitol, mannitol, alditol, pantothenol or mixtures thereof. Their amount used in the preparation according to the invention is in that case between 0.3 and 10% by weight, preferably between 1 and 5% by weight. By virtue of their hygroscopic properties, the divalent or polyvalent alcohols usually serve in cosmetic preparations as moisture donors. In the preparation according to the invention, by virtue of their relatively very low evaporation rate, they serve as permanent plasticisers which reliably prevent the applied material from flaking or crumbling off. Because of their hygroscopic properties it is particularly surprising that they do not in any way adversely affect the water-resistance and tear-resistance of the preparation according to the invention.

A further essential component of the cosmetic preparation according to the invention is a polymeric film-forming system. The film-forming system is usually formed from a polymer or a plurality of polymers which is/are present in a medium in the form of a dispersion or a solution, wherein the polymer or polymers forms or form a film upon the removal of that medium, that is to say the solvent or dispersing agent respectively. Depending on the kind of polymer the solid content is between 10 and 70% by weight, preferably between 20 and 50% by weight. In individual cases the polymers or polymer mixtures can also be in the form of solubilisable polymer powders. In the present application any system comprising film-forming agent and medium (also referred to as solubilisation agent, solvent or dispersing agent) is identified generally as ‘dispersion’ to avoid lack of clarity. As is known, the transitions between a dispersion and a solution are fluid in the field of polymers. Therefore the expression ‘dispersion’ also embraces solutions. Also included are systems in which the film-forming polymer was solubilised for example by the addition of an acid or base. The medium is preferably water.

Film-forming polymers are known per se and all those usually employed in cosmetic preparations can be used. The film-forming agent, together with the wax component, results in a very durable, water-resistant and tear-resistant coating for the keratinic material, for example for eyelashes, eyebrows, hair, hair piece, hair at the temples or beard.

The film-forming polymer can be produced by polyaddition, polycondensation or polymerisation or can be in the form of a polymer of natural origin. Particularly preferred in accordance with the invention are polyvinyl alcohols, PVP/alkene copolymers and functionalised polyurethanes, in particular polyurethanes hydrophilised by the incorporation of inorganic and/or organic acid esters. Sulfonate, sulfate, phosphonate, phosphate and carboxylate residues are to be listed by way of example as acid residues.

The film-forming system is used in an amount which produces the desired effect. In that respect amounts in the range of between 0.1 and 50% by weight are suitable. In order to achieve particularly advantageous properties in regard to durability, adhesion to the selected substrate and flexibility and shaping force, the amounts used are in the range of between preferably 0.3 and 35% by weight and particularly preferably between 1 and 25% by weight, wherein, in an individual case, the amount depends on the polymer used and the proportion and nature of the wax component and the gel component. The stronger the structure formed by the gel component or the stronger the respective film formed, the correspondingly smaller can be the amount of film-forming system. The optimum amount can be easily established by the man skilled in the relevant art. In this respect the quantitative specification relates to the system used, that is to say the amount of film-forming polymers and solubilisation agent.

A further component, which is essential to the invention, of the preparation, in particular the cosmetic preparation, is a gel component which must be included in order to achieve the advantageous properties. It was found that the use of a pre-swollen hydrocolloid as a gel component has a structure-forming action and stabilises the structure in such a way that after drying the composition forms a permanent, flexible and water-resistant and tear-resistant film on keratinic material and, together with the film-forming agent and the wax component, retains the further ingredients of the preparation according to the invention in that composition in such a way that they are not leached out and cannot migrate or bleed out. The gel component therefore provides a material which, as stated above, is ideally suitable for receiving and retaining coloring agents and pigments but also further ingredients.

The gel component is formed from at least one pre-swollen hydrocolloid, swelling preferably being effected in water. It was found that a hydrocolloid in the swollen condition produces the desired lattice which then, in conjunction with the film-forming system, can accommodate and stabilise other ingredients. In connection with the present invention, those water-soluble, natural or synthetic polymers which form gels or viscous, in particular highly viscous solutions, in solvents, in particular aqueous systems, are to be identified as hydrocolloids. Examples of possible gel-forming agents are basically known in powder form and as thickening agents in cosmetic preparations—mention is to be made here by way of example of celluloses and cellulose derivatives, starch and starch derivatives, alginates, pectins, carrageenans, tragacanth, plant rubbers, polyvinyl alcohol, polyvinyl pyrrolidone and dextran and mixtures thereof. Hitherto however it was not realised that they can impart very advantageous properties to a cosmetic preparation, in the pre-swollen condition. In that respect, a combination of polyvinyl alcohol and hydroxyethyl cellulose has surprisingly been found to be particularly advantageous, if in that respect a quantitative ratio of between 10 and 1 and in particular between 6 and 1 is adopted. It was found in that case that this combination of two hydrocolloids in the pre-swollen condition imparts structure, stability and volume to the preparation according to the invention. The use of the combination according to the invention of two pre-swollen hydrocolloids can even have the result that the volume of the preparation is scarcely reduced or not reduced at all, after drying. In that way the preparation according to the invention, even after drying, makes keratinic materials, in particular eyelashes, fuller and longer, imparts a curve thereto and thus enhances the esthetic impression.

It is essential in that respect that the combination of the hydrocolloids—added individually or jointly, for the avoidance of process mistakes, to the later preparation—is used in the fully swollen condition, that is to say in the form of a stable aqueous gel or a highly viscous aqueous solution, for the production of the preparation according to the invention, and is not for example only allowed to swell in the preparation. Particularly good properties are achieved if the combination of the hydrocolloids is involved in heated water, preferably in water at a temperature of between 40 and 90° C. Usually the combination of the hydrocolloids—or each in itself—is strewn with agitation into water and then, after complete swelling, prior to further processing, allowed to cool preferably to about 40° C., particularly preferably to room temperature. Due to the swelling effect the physical properties of the hydrocolloids are irreversibly changed, and that affords the desired structure. The resulting hydrocolloid or the combination of two or more hydrocolloids respectively is therefore stable and storable and can therefore also be stored for some time, for example some hours or even over still longer periods of time, prior to further processing. To avoid microbial attack on the pre-swollen hydrocolloid system during storage, it is possible to add a preserving agent. Preserving agents for systems of that kind are known to the man skilled in the relevant art. The preserving agents which are approved and suitable for foods and cosmetics can be employed for that purpose.

In addition a dispersing additive can also be added to the system. It was found that a combination of preserving agent and dispersing additive can synergistically enhance the anti-microbial effect. In that connection an addition of EDTA and salts thereof also has an advantageous effect as calcium ions are complexed in that way, which makes the cell membranes of micro-organisms more permeable for preserving agent. Benzyl alcohol, phenethyl alcohol, phenoxy ethanol, esters of p-hydroxybenzoic acid and salts thereof, IPBC, formaldehyde splitters such as imidazolidinyl urea or diazolidinyl urea, organic and organic aromatic carboxylic acids and mixtures of the specified substances can be mentioned as suitable preserving agents. In that respect it is to be noted that their use and the maximum amounts are also permitted by the respective national or regional legislation.

The preparation according to the invention may also contain still further usual cosmetic ingredients such as antioxidants, fragrances, vitamins, extracts, active substances, protein hydrolysates and other additives which influence certain desired properties. An important ingredient for cosmetic preparations, in particular for decorative cosmetic preparations, are coloring agents, that is to say dyes or pigments, in order to give a desirable color impression. Besides those, it is also possible to use fillers in the form of preferably finely divided, inorganic or organic solids. It was found that the preparation according to the invention is suitable for stabilising both fillers such as talcum, kaolin, healing earth, smectite, bentonite, hectorite, montmorillonite, starch and modified starch, non-swelling starches, water-insoluble celluloses, polytetrafluoroethylene powder (Teflon), polyamide powder (Nylon), polyethylene powder, polypropylene powder, silk powder, silicon dioxide, boronitride, mica, fine fiber material of silk, cotton, wool, linen, polyamide, polyacrylnitrile, polyesters, elastanes, viscose, artificial silk, cellulose or the like, insoluble metal soaps such as Mg-stearate, Ca-stearate, Sr-stearate or Zn-stearate or mixtures thereof and/or for including and dispersing inorganic or organic pigments and lakes of organic coloring agents in such a way that they cannot be leached out or emigrate or bleed out. All coloring agents which are approved for cosmetics by the respective national or regional legislation can be used as the coloring agents. Dyes are in that respect generally less advantageous because under some circumstances they can color keratinic materials semi-permanently, that is to say over a period of several days; use thereof is however not basically excluded as a result, as that can certainly also be a desired effect. Preferably pigments are used for coloring the preparation according to the invention, in micronised form. In quite general terms the expression ‘pigments’ is used to denote white or colored, inorganic or organic particles which are insoluble in water and the respective medium and which can color and/or cloud a composition. This can involve white or colored, inorganic or organic pigments of commercially available size or with particles sizes in the nanometer range, that is to say so-called ‘nanopigments’. Ultrafine aluminum oxide, cerium oxide, titanium oxide, zinc oxide or mixtures thereof which have average particles sizes in the range of between 5 and 25 nm are to be mentioned in that area. The man skilled in the relevant art implements the choice of suitable coloring agents in accordance with the desired effects and within the limits of the statutory requirements. Examples of suitable inorganic and organic pigments are titanium dioxide, zinc oxide, barium sulfate, carbon black, carmine, yellow, red or black iron oxide, chrome oxide green, hydrated chrome oxide green, ultramarine, Berlin Blue (Ferric Blue), manganese violet. Examples of organic coloring agents are lakes of organic dyes such as aluminum, barium, calcium, potassium, strontium or zirconium lake and other substances which are well-known to the man skilled in the relevant art or mixtures of the specified substances. In addition it is also possible to use pearlescent pigments and other ‘glitter substances’ such as titanium dioxide-coated mica and colored mica coated with titanium dioxide and metal oxides, bismuth oxychloride, bismuth oxychloride coated with metal oxides, flake-form metal powders of aluminum, bronze, brass, copper, silver and gold. Also suitable are ‘glitter substances’ based on fine flake-form glasses which can possibly also be at least partially coated with titanium dioxide and/or colored metal oxides, flake-form metal powders at least partially coated with other materials, plastic flakes at least partially coated with other materials, preferably PET flakes, solid solutions of dyes in solid plastic matrices, preferably polyester-3 or mixtures of the above-mentioned colored substances. The last-mentioned solid solutions of dyes in plastic matrices which are distinguished by intensively lighting up in long-wave UV-light, so-called ‘black light’, are commercially inexpensive and known for example under the INCI names ‘Polyester-3, Red 22’, ‘Polyester-3, Red 28’, ‘Polyester-3, Yellow 10’ or ‘Polyester-3, Blue 1’, see in that respect for example also WO 2004/108108. They can also be used in mixtures with each other, possibly also in conjunction with UV-active stilbene derivatives which are allowed for cosmetic uses and which are distinguished in long-wave UV-light by an intensive light-blue lighting effect or in any mixtures with the above-mentioned coloring agents which can also be in a form which is surface-coated with silicones or fatty acids, for example hydrophobised, according to the desired color effects. That list is only by way of example and is in no way definitive.

If the preparation according to the invention is used as mascara, generally those pigments are used, which result in the colors that are usually wanted, namely black, brown, blue and gray. Nonetheless it is however also possible to produce preparations of other colors in order to fashionably accentuate and give high-contrast coloring to the eyelash tips or the eyelashes also overall with contrasting colors, for example light green, moss-green, yellow, orange or different shades of red. Particularly in the field of hair mascara, the man skilled in the art, based on the above-listed coloring agents, is presented with a wide range of possible options including for the sector of so-called ‘effect makeup’, for example in regard to colors for the consumers to visit a disco.

The amount of the finely divided solids phase comprising inorganic and/or organic fillers and inorganic and/or organic coloring agents is generally in the range of between 0.1 and 30% by weight, preferably between 1 and 20% by weight and particularly preferably between 5 and 15% by weight.

The coloring constituents do not necessarily have to be contained in the preparation according to the invention. It is also possible to have an embodiment in which coloring and structuring constituents are separate, wherein firstly the preparation according to the invention is applied without coloring ingredients to the keratinic material and then a formulation which contains the coloring agents is applied. It is also possible for in particular large-area ‘glitter substances’ which can be of average particle sizes in the range of between about 50 to 500 μm to be suitably subsequently applied in dry form to the preparation according to the invention which is not colored and when it has not yet dried.

The pH-value of the preparation according to the invention should preferably be in the neutral to weakly basic range as tear fluid does in fact have a pH-value of about 7.4 and, when using organically neutralised fatty acid, it is only in that medium that it can deploy its full effect as a co-emulsifier and emulsion stabiliser. Optionally, to adjust and buffer the pH-value the preparation according to the invention also contains basic agents and/or buffering agents, for example soda lye or potash lye, amines and conventional buffer substances. Preferably the pH-value of the preparation is so adjusted that it is in the range of between 6.5 and 8.0 and preferably between 6.8 and 7.5. According to the invention therefore there is provided a preparation which is in the form of an emulsion and which combines particularly advantageous properties. The preparation can be easily produced, it leads to stable products, it can be easily subjected to further processing as it is stable in the hot condition. It produces a film which adheres for a long period of time without liberation of the ingredients contained therein. The film causes the keratin fibers to which it is applied to look full and gives them shape and curve—it ‘curls’ them therefore in an attractive and permanent fashion. At the desired time the preparation can also be easily removed again by applying water, possibly slightly heated and provided with a wetting agent, which results in swelling of the film whereby it can be easily removed again without leaving any residue. The product according to the invention however can equally well be removed again with a commercially available makeup remover or suitable cloth. The specific combination of wax component, polyvalent alcohol, film-forming agent and gel component forms a network or lattice which adheres durably to the keratin fiber so that the result obtained is a homogeneous, bulky, flexible film which lasts for a long time.

To produce the preparation according to the invention, the aqueous gel component is prepared by the combination of at least two hydrocolloids—which are present mixed jointly or in succession—is pre-swollen in a partial amount of the water required for production of the preparation. For that purpose preferably heated water which is advantageously at a temperature of between 40 and 90° C. and preferably between 60 and 80° C. is brought into contact with the hydrocolloids with agitation and then thereafter possibly allowed to cool and swell. The at least divalent, polyvalent alcohol or a mixture of two or more of those alcohols is then added to that mixture. A preserving agent and/or a dispersing additive and/or the base required for at least partial neutralisation of the fatty acid can optionally also be added to that mixture—the base however can equally well be added to the remaining amount of water, which is still present, at a later time, that is to say immediately prior to production of the emulsion. The swollen gel is stable and can be stored even over prolonged periods of time so that it does not have to be subjected to further processing immediately.

The constituents of the wax component are weighed out into a suitable vessel and heated together to between 50 and 100° C., preferably between 60 and 80° C. The remaining amount of water is now heated to about 5° C. above the temperature of the melted wax component, then the necessary amount of base is added, then the liquid wax component is added to the water with vigorous agitation. To provide for intensive thorough mixing of the O/W emulsion which is being formed, a high-speed agitator is preferably used or the entire production procedure is implemented in an evacuatable process installation. The composition is now left to cool to about between 50 and 45° C., the swollen gel phase is added and the composition is now homogenised for several minutes. The solids phase comprising pigments and optionally fillers is then added and once again thoroughly homogenised. Now the temperature-sensitive substances such as for example antioxidants, fragrance mixtures, finished polymer dispersions, volatile substances such as silicone oils or isoparaffins and the like are added and the finished preparation is then cooled either to room temperature and filled into fixedly closable storage containers or at a temperature of about 40° C. conveyed away directly to the filling operation and filled into suitable, commercially available application devices. The process according to the invention makes it possible for the preparation to be manufactured easily, in a well-reproducible fashion and rationally in large numbers. The filling operation while still in the heated condition in accordance with the process of the invention thus affords advantages both from the point of view of hygiene and also from the point of view of process engineering.

Conventional application devices for mascara—they are referred to hereinafter as ‘containers’ and usually have a receiving space comprising a transparent material or a material which is colored with the color of the material or the ‘corporate color’ of the supplier, wherein the receiving space is provided with a releasably mounted closure on which there is disposed an inwardly extending stem carrying a brush for application of the material to be applied—are commercially available and are sufficiently known to the man skilled in the art—here he only has to take account of the fact that the selected brush provides for a good wetting effect for the eyelashes and in so doing separates them and that the brush and the scraper portion and in addition the material of the container and the ingredients of the mascara preparation are well matched to each other. If a mascara preparation and an effect mascara are offered in combination, a packaging which can be combined from two individual units is then recommended—however even those combination packagings in which one unit can be provided with a smaller volume and a smaller brush have in the meantime already become commercially available. As normally a person has more hair on the head than eyelashes, a larger container naturally has to be adopted for hair mascara. The preparation according to the invention, in particular the cosmetic preparation, is particularly well suited to coating keratinic materials. It is therefore preferably used for coloring and/or shaping eyelashes, eyebrows, hair, hair pieces, hair which has turned gray at the temples, beard hair and other keratinic materials. The preparation according to the invention is particularly suitable as mascara as it clings firmly to the eyelashes, covers them well and uniformly, it does not migrate out, it does not come off and it does not bleed out, it does not crumble away and it does not flake off and it remains on the eyelash for a long time and in a durable state until it is removed again. As it has a smaller proportion of solids than previously known products, it is forgiving in regard to mistakes in use and allows subsequent corrections. The volatile component contained in the preparation according to the invention causes minimal surface dissolution of the film present on the eyelash and thus results in an intimate join between material which is already present there and freshly applied material. In that respect drying of the preparation takes place with something of a delay and in two steps as firstly the water and then the volatile component evaporates, which in turn is conducive to the ‘curl’ of the applied film.

A further subject of the invention is therefore the use of the preparation according to the invention for coloring and shaping keratinic material such as eyelashes, eyebrows, hair, hair pieces, hair which has turned gray at the temples, beard hair and other keratinic materials, in particular use as mascara.

### Embodiments

The invention is described in detail by the Examples hereinafter without however being restricted thereto. In that respect the amounts are given in percent by weight (% by weight), in each case with respect to the total weight of the preparation. The raw materials are identified by use of the ‘INCI’ names which are generally familiar to the man skilled in the relevant art—‘INCI’ is the ‘International Nomenclature of Cosmetic Ingredients’.

### EXAMPLE 1

### Pasty Mascara Preparation

| (A) | |
|---|---|
| Aqua (freshly distilled water) | 51.380 |
| Polyvinyl alcohol | 6.000 |
| Hydroxyethyl cellulose | 1.300 |
| Sorbitol | 2.500 |
| Glycerin | 1.000 |
| Panthenol | 0.250 |
| Aminomethyl propanol | 0.550 |
| (B) | |
| Sorbitan olivate | 6.500 |
| Stearic acid | 4.000 |
| PVP/hexadecene copolymer | 1.500 |
| Copernicia cerifera (carnauba) | 3.500 |
| Oryza sativa (rice bran wax) | 4.500 |
| Isopropyl myristate and bambusa vulgaris extract | 0.200 |
| Bis-PEG/PPG-16/16 PEG/PPG-16/16 dimethicone | 0.500 |
| Caprylic/capric triglyceride | 0.300 |
| (C) | |
| Iron oxide black (C.I. No 77.499) | 9.500 |
| Tocopherol acetate | 0.350 |
| Fragrance | 0.250 |
| Dimethicone 1,5 st | 4.500 |
| Polyurethane-2 | 1.000 |
| IPBC | 0.020 |
| Phenoxyethanol | 0.400 |

For the production procedure approximately half of the above-specified amount of water is put into a sufficiently large vessel and heated to about 65° C. The polyvinyl alcohol and the hydroxyethyl cellulose are then added with vigorous agitation using a toothed ring agitator and agitated to a lump-free condition. The mixture is left to cool to about 45° C. and now sorbitol, glycerin and panthenol are added. IPBC and phenoxyethanol can possibly also be added. The constituents of phase (B) are heated in a separate vessel to about 70° C. The balance of water from phase (A) is heated to about 75° C., the aminomethyl propanol is added and the mixture is transferred into a vacuum process installation. The liquid phase (B) which has been heated to about 70° C. is now added, a slight vacuum is applied, the mixture is homogenised briefly and cooled to about 45° C. The mixture is now homogenised for some minutes under vacuum, the pigment is added, the mixture is homogenised once again briefly under vacuum and now the swollen gel phase is added. The mixture is now once again homogenised briefly under vacuum, the constituents which have not yet been added are now added, the pH-value is adjusted to between 7.2 and 7.4 and then the mixture is selectively either cooled to room temperature or the composition is filled in a condition of still being heated into sealingly closable storage containers or it is prepared for filling into conventional application devices. The product obtained is a deep black pasty preparation with a good sheen, which can be very easily and uniformly distributed on keratinic material, which has extremely good adhesion thereto and which is completely dried relatively quickly.

### EXAMPLE 2

### Pasty, Colorless Mascara Preparation

| (A) | |
|---|---|
| Aqua (water, freshly distilled) | 56.830 |
| Polyvinyl alcohol | 5.500 |
| Carrageenan | 1.500 |
| Mannitol | 2.000 |
| Propylene glycol (propan-1,2-diol) | 1.700 |
| Aminomethyl propanol | 0.650 |
| (B) | |
| Sorbitan olivate | 6.500 |
| Behenic acid | 4.200 |
| PVP/eicosene copolymer | 1.900 |
| Euphorbia cerifera (candellila wax) | 4.500 |
| Behenyl behenate | 4.000 |
| Bis-PEG/PPG-16/16 PEG/PPG-16/16 dimethicone | 0.500 |
| Octyldodecanol | 0.300 |
| (C) | |
| Tocopherol acetate | 0.350 |
| Fragrance | 0.250 |
| Isopropyl myristate and bambusa vulgaris extract | 0.200 |
| Dimethicone 1,5 st | 6.500 |
| Polyurethane-2 | 2.200 |
| IPBC | 0.020 |
| Phenoxy ethanol | 0.400 |

Production and processing are effected in a similar manner to Example 1, with omission of the step of pigment incorporation. The result obtained is a shiny material which is colorless after drying and which can be applied to eyelashes to impart fullness and curl thereto. When applied to the air, before the film dries, coarser particles of a ‘glitter substance’, for example colored polyester particles, can be scattered thereonto.

### EXAMPLE 3

### Pasty Effect Hair Mascara

| (A) | |
|---|---|
| Aqua (water, freshly distilled) | 50.750 |
| Polyvinyl alcohol | 5.000 |
| Tragacanth | 2.000 |
| Xylitol | 2.500 |
| Dipropylene glycol | 1.800 |
| Triethanolamine | 0.750 |
| (B) | |
| Sorbitan olivate | 5.500 |
| Isostearic acid | 4.500 |
| PVP/eicosene copolymer | 1.800 |
| Copernicia cerifera (carnauba) | 4.000 |
| Ouricuri wax | 3.500 |
| Bis-PEG/PPG-16/16 PEG/PPG-16/16 dimethicone | 0.600 |
| Butyl stearate | 0.400 |
| (C) | |
| Polyester-3, Red 28 | 5.500 |
| Titanium dioxide (C.I.-No 77891), nanopigment | 2.500 |
| Tocopherol acetate | 0.200 |
| Isopropyl myristate and bambusa vulgaris extract | 0.200 |
| Fragrance | 0.150 |
| Decamethylcyclopentasiloxane | 5.500 |
| Polurethane-2 | 2.200 |
| Methylparaben | 0.150 |
| Propylparaben | 0.100 |
| Phenoxyethanol | 0.400 |

Production is effected in a similar manner to Example 1. The result obtained is a shiny, magenta-colored, pasty preparation which can be easily and uniformly applied to the hair and which is preferably suitable for coloring streaks. In long-wave UV light the preparation has intensive red-violet fluorescence.

### EXAMPLE 4

### Comparative Example—Mascara

| (A) | |
|---|---|
| Aqua (water, freshly distilled) | 57.550 |
| Acacia senegal gum | 2.000 |
| Hydroxyethyl cellulose | 1.300 |
| Polyvinyl alcohol | 4.000 |
| Triethanolamine | 0.650 |
| Panthenol | 0.250 |
| (B) | |
| Hydrogenated stearyl olive esters | 5.000 |
| Stearic acid | 3.000 |
| Palmitic acid | 2.500 |
| Oryza sativa (rice bran wax) | 4.500 |
| Copernica cerifera (carnauba) | 1.500 |
| Euphorbia cerifera (candelilla wax) | 2.000 |
| Dimethicone copolyol | 1.500 |
| Simethicone | 1.000 |
| (C) | |
| Black iron oxide (C.I.-No 77499) | 9.500 |
| Mica (C.I.-No 77891) | 1.500 |
| Methylparaben | 0.150 |
| Propylparaben | 0.100 |
| PPG-17/PDI/DMPA copolymer | 2.000 |

Production is effected in a similar manner to Example 1; the preparation however is cooled in conventional manner to room temperature after a partial amount discharged at 40° C., after cooling, exhibited a gritty structure. The result obtained is a deep black, pasty, shiny material which can be applied well and uniformly to the eyelashes and which clings firmly there. In an attempt to apply the material once again to an eyelash which had already been previously colored with the same material, that results in an irregular appearance involving irregular structures. The material has a dank, musty odor of its own. Stored in a closed glass container over 24 hours at 50° C., the material assumes a gritty structure and at the edge exhibits syneresis and liquid deposits on the surface. The surface has a dull and very non-homogeneous effect. In a preservation stress test, after just a single inoculation, the material exhibited massive growth with mold and yeast.

1. A cosmetic preparation in the form of an O/W emulsion, comprising a wax component, a fatty acid, at least one polyvalent alcohol, at least one film-forming agent, a non-ionogenic emulsifier and at least one gel component, wherein the gel component comprises has at least one swollen hydrocolloid.

2. A preparation as set forth in claim 1, wherein the wax component comprises one or more waxes which are solid at room temperature and have a dropping point of between 45 and 200° C.

3. A preparation as set forth in claim 1, wherein the wax component comprises one or more waxes which are solid at room temperature and have a hardness of between 2 and 40, determined using the needle penetration method in accordance with ASTM D5.

4. A preparation as set forth in claim 2, wherein the wax component comprises waxes selected from the group consisting of vegetable, animal, mineral, synthetic and mixtures thereof.

5. A preparation as set forth in claim 4, wherein the wax is selected from the group consisting of carnauba wax, candelilla wax, ouricuri wax, Japan wax, cotton wax, rice wax, flower wax, fruit wax, hydrated jojoba oil, beeswax, polyglyceryl-3 beeswax, lanolin wax, montan wax, ozocerite, paraffin wax, microcrystalline wax, Fischer-Tropsch wax, polyethylene wax, silicone wax, wax polymer hybrid, cetyl palmitate, cetyl stearate, behenyl stearate, behenyl oleate, behenyl behenate, acelaic acid dioleylester, acelaic acid dibehenylester and mixtures thereof.

6. A preparation as set forth in claim 1, wherein the wax component comprises a combination of an emulsifier and a co-emulsifier.

7. A preparation as set forth in claim 6, wherein the emulsifier is a non-ionogenic emulsifier.

8. A preparation as set forth in claim 7, wherein the emulsifier is a non-ionogenic emulsifier which has no PEG or PPG residues.

9. A preparation as set forth in claim 6, wherein the emulsifier is sorbitan olivate.

10. A preparation as set forth in claim 9, wherein the co-emulsifier is a fatty acid at least partially neutralised with an organic base.

11. A preparation as set forth in claim 10, wherein the organic base is aminomethyl propanol or triethanolamine or mixtures thereof.

12. A preparation as set forth in claim 11, wherein the at least partially neutralised fatty acid is a saturated, straight-chain or branched-chain fatty acid.

13. A preparation as set forth in claim 10, wherein the at least partially neutralised fatty acid is selected from the group consisting of is palmitic acid, stearic acid, isostearic acid, behenic acid and mixtures thereof.

14. A preparation as set forth in claim 10, wherein the at least partially neutralised fatty acid is stearic acid.

15. A preparation as set forth in claim 14, wherein the stearic acid is at least partially neutralised with aminomethyl propanol.

16. A preparation as set forth in claim 14, wherein the ratio of sorbitan olivate to stearic acid is between 3 to 1 and 1 to 3.

17. A preparation as set forth in claim 16, wherein a PVP copolymer is additionally used as a further co-emulsifier.

18. A preparation as set forth in claim 17, wherein the PVP copolymer is a PVP/alkene copolymer.

19. A preparation as set forth in claim 18, wherein the PVP/alkene copolymer is selected from the group consisting of a PVP/hexadecene copolymer, a PVP/eicosene copolymer, and mixtures thereof.

20. A preparation as set forth in claim 17, wherein the quantitative ratio between sorbitan olivate and PVP copolymer is between 10 to 1 and 3 to 1.

21. A preparation as set forth in claim 1, wherein the at least one polyvalent alcohol comprises at least divalent alcohol.

22. A preparation as set forth in claim 21, wherein the at least divalent alcohol has functional groups.

23. A preparation as set forth in claim 22, wherein the at least divalent alcohol is selected from the group consisting of propane diol, dipropylene glycol, tripropylene glycol, glycerin, diglycerin, triglycerin, butane diols, pentaerythritol, hexane diols, sorbitol, xylitol, mannitol, alditol, pantothenol and mixtures thereof.

24. A preparation as set forth in claim 23, wherein the amount of the at least divalent alcohol used is between 0.3 and 10% by weight, with respect to the total weight of the preparation.

25. A preparation as set forth in claim 23, wherein the amount of the at least divalent alcohol used is between 1 and 5% by weight, with respect to the total weight of the preparation.

26. A preparation as set forth in claim 1, wherein the film-forming agent is a film-forming polymer which is dispersed and/or dissolved in a medium.

27. A preparation as set forth in claim 26, wherein the film-forming agent is selected from the group consisting of polyvinyl alcohol, PVP/alkene copolymer, a functionalised polyurethane and mixtures thereof.

28. A preparation as set forth in claim 27, wherein the functionalised polyurethane is a hydrophilised polyurethane into which inorganic and/or organic acid residues are incorporated.

29. A preparation as set forth in claim 28, wherein the organic acid residues are selected from the group consisting of sulfonate, sulfate, phosphonate, phosphate, carboxylate and mixtures thereof.

30. A preparation as set forth in claim 28, wherein the functionalised polyurethane is polyurethane-2.

31. A preparation as set forth in claim 26, wherein the film-forming agent is used in amounts of between 0.1 and 50% by weight, with respect to the total weight of the preparation.

32. A preparation as set forth in claim 26, wherein the film-forming agent is used in amounts of between 0.3 and 35% by weight, with respect to the total weight of the preparation.

33. A preparation as set forth in claim 26, wherein the film-forming agent is used in amounts of between 1 and 25% by weight, with respect to the total weight of the preparation.

34. A preparation as set forth in claim 1, wherein the at least one swollen hydrocolloid is a water-soluble natural or synthetic polymer which swells in water and forms a gel or a highly viscous solution.

35. A preparation as set forth in claim 1, wherein the at least one swollen hydrocolloid is selected from the group consisting of celluloses, cellulose derivatives, starch, starch derivatives, alginates, pectins, carrageenans, tragacanth, plant rubbers, polyvinyl alcohol, polyvinyl pyrrolidone, dextran and mixtures thereof.

36. A preparation as set forth in claim 1, wherein the at least one gel component comprises an aqueous gel or a highly viscous aqueous solution of the at least one swollen hydrocolloid.

37. A preparation as set forth in claim 36, wherein the aqueous gel or the highly viscous aqueous solution is selected from the group consisting of a water-swollen polyvinyl alcohol, hydroxyethyl cellulose, carrageenan, tragacanth and mixtures thereof.

38. A preparation as set forth in claim 36, wherein the aqueous gel or the highly viscous aqueous solution comprises a water-swollen polyvinyl alcohol in combination with at least one of hydroxyethyl cellulose, carrageenan, and tragacanth.

39. A preparation as set forth in claim 36, wherein the aqueous gel or the highly viscous aqueous solution comprises at least one of a preserving agent, at least one dispersing additive, and at least one plasticiser.

40. A preparation as set forth in claim 1, wherein the preparation additionally contains a finely divided solids phase.

41. A preparation as set forth in claim 40, wherein the finely divided solids phase is selected from the group consisting of inorganic fillers, organic fillers, fine fiber materials, inorganic pigments, organic pigments and mixtures thereof.

42. A preparation as set forth in claim 41, wherein the fillers is selected from the group consisting of talcum, kaolin, healing earth, smectite, bentonite, hectorite, montmorillonite, starch and modified starches, non-swelling starches, water-insoluble celluloses, tetrafluoroethylene powder, polyamide powder, polyethylene powder, polypropylene powder, silk powder, silicon dioxide, boronitride, mica, fiber material, insoluble metal soaps and mixtures thereof.

43. A preparation as set forth in claim 41, wherein the fiber material is selected from the group consisting of silk, cotton, wool, linen, polyamide, polyacrylonitrile, polyesters, elastanes, viscose, artificial silk, cellulose and mixtures thereof.

44. A preparation as set forth in claim 42, wherein the insoluble metal soaps are selected from the group consisting of Mg stearate, Ca stearate, Sr stearate, Zn stearate and mixtures thereof.

45. A preparation as set forth in claim 41, wherein the inorganic pigments and organic pigments are selected from the group consisting of white and/or colored pigments of commercially available size or of particle sizes in the nanometer range.

46. A preparation as set forth in claim 41, wherein the pigments are white pigments in the nanometer range and are selected from the group consisting of aluminum oxide, cerium oxide, titanium dioxide, zinc oxide and mixtures thereof, and have an average particle size of between 5 and 25 nm.

47. A preparation as set forth in claim 41, wherein the pigments comprise titanium dioxide, zinc oxide, barium sulfate, carbon black, carmine, iron oxides, chrome oxide green, hydrated chrome oxide green, ultramarine, Berlin Blue, manganese violet, and lakes of organic coloring agents, including aluminum, barium, calcium, potassium, strontium, zirconium lakes and mixtures thereof.

48. A preparation as set forth in claim 41, wherein the pigments comprise ‘glitter substances’ selected from the group consisting of mica coated with titanium oxide and/or metal oxides, bismuth oxychloride, bismuth oxychlorides coated with metal oxides, flake-form metal powders, flake-form metal powders at least partially coated with other materials, flake-form glasses, plastic flakes at least partially coated with other materials and/or solid solutions of dyes in plastic matrices and mixtures thereof.

49. A preparation as set forth in claim 48, wherein the solid solution of dyes in plastic matrices is a solution of dye in polyester-3.

50. A preparation as set forth in claim 49, wherein the solid solution of the dye in polyester-3 exhibits intensive lighting in long-wave UV light.

51. A preparation as set forth in claim 50, wherein the solid solution of a dye in polyester-3 is selected from the group consisting of ‘Polyester-3, Red 22’, ‘Polyester-3, Red 28’, ‘Polyester-3, Yellow 10’, ‘Polyester-3, Blue 1’, and mixtures thereof.

52. A preparation as set forth in claim 40, wherein the amount of the finely divided solids phase in the preparation is between 0.1 and 30% by weight.

53. A preparation as set forth in claim 40, wherein the amount of the finely divided solids phase in the preparation is between 1 and 20% by weight.

54. A preparation as set forth in claim 40, wherein the amount of the finely divided solids phase in the preparation is between 5 and 15% by weight.

55. A preparation as set forth in claim 1, wherein the preparation further comprises at least one of preserving agents, antioxidants, vitamins, fragrance mixtures, extracts, active substance, protein hydrolysates, polymer dispersions, volatile substances and adjuvants.

56. A preparation as set forth in claim 55, wherein the volatile substances are selected from the group consisting of silicone oils, paraffins, isoparaffins and mixtures thereof.

57. A preparation as set forth in claim 55, wherein the volatile substances comprise silicones selected from the group consisting of linear silicone oils, cyclic silicone oils, and mixtures thereof.

58. A preparation as set forth in claim 57, wherein the volatile silicones are selected from the group consisting of hexamethyl disiloxane, octamethyl trisiloxane, decamethyl tetrasiloxane, dodecamethyl pentasiloxane, hexamethylcyclo trisiloxane, octamethylcyclo tetrasiloxane, decamethylcyclo pentasiloxane, dodecamethylcyclo hexasiloxane and mixtures thereof.

59. A preparation as set forth in claim 56, wherein the paraffins and/or isoparaffins are selected from the group consisting of decane, isodecane, dodecane, isododecane, tetradecane, isotetradecane and mixtures thereof.

60. A preparation as set forth in claim 59, wherein the paraffins are C₁₁/C₁₂ isoparaffin in a mixture with volatile silicone oils.

61. A process for the production of a gel-bearing preparation, in particular a cosmetic preparation, wherein a combination of at least two hydrocolloids is swollen in water optionally in the presence of a preserving agent and/or a dispersing additive and the mixture is then mixed with at least one at least divalent alcohol; the wax component is melted, an emulsion is formed from wax component and water to which the base necessary for partial neutralisation of the fatty acid was added, the combination of the swollen hydrocolloids is added to the emulsion, and the mixture is agitated until it is homogeneous and then the remaining ingredients including the finely divided solids phase are added and the preparation is again agitated until it is homogeneous.

62. A process as set forth in claim 61, including adjusting the pH-value of the mixture to between 6.5 and 8.

63 A process as set forth in claim 61, including adjusting the pH-value of the mixture to between 6.9 and 7.5.
